(19) United States
(12) Patent Application Publication
(10) Pub. No.: US 2019/0053924 A1
(43) Pub. Date: Feb. 21, 2019

(54) STENT AND STENT MANUFACTURING METHOD

(71) Applicant: GWANGJU INSTITUTE OF SCIENCE AND TECHNOLOGY, Buk-gu, Gwangju (KR)

(72) Inventors: Woorim CHOI, Buk-gu, Gwangju (KR); Yonggu LEE, Buk-gu, Gwangju (KR)

(73) Assignee: GWANGJU INSTITUTE OF SCIENCE AND TECHNOLOGY, Buk-gu, Gwangju (KR)

(21) Appl. No.: 15/764,190

(22) PCT Filed: Oct. 28, 2016
(86) PCT No.: PCT/KR2016/012269

§ 371 Date: Mar. 28, 2018

(30) Foreign Application Priority Data
Oct. 28, 2015 (KR) 10-2015-0150562

(51) Int. Cl. A61F 2/90

(52) U.S. Cl. CPC A61F 2/90; A61F 2002/048

(57) ABSTRACT

The present disclosure provides a stent comprising: a hollow tubular body portion; a hooking portion connected to one end of the body portion; and a hooked portion connected to the other end of the body portion, wherein the hooking portion is hooked on the hooked portion. According to the present disclosure, the stent may be manufactured by 4D printing method. Accordingly, the stent may be manufactured in an automated process at low cost, expeditiousness, simplicity, and no manufacturing site constraints.

### TECHNICAL FIELD

The present disclosure relates to a stent and a method for manufacturing the stent.

### BACKGROUND ART

The stent refers to a tube, or other device similar to a tube, that is inserted into the body to connect two hollow sections together. For example, the stent is inserted into the blood vessels and ureters in the human body, thereby forming passage extension of blood vessels and ureters.

There are two main methods for manufacturing the stent. The first method is a method of fabricating a net-like stent by weaving wires. The second method is to fabricate the mesh of the stent by using a laser processing method. The prior art of the first method is disclosed in Korean Patent Laid-Open Publication No. 10-2013-0045977. The stent manufacturing method is complicated. In particular, the surface of the stent must be smooth so that bleeding or inflammatory reaction may be suppressed after the stent is inserted into the body. Thus, the stent manufacturing process becomes more complicated due to the post-process such as the chemical treatment or the electrolytic polishing process to allow the stent surface to be smooth.

As a result, the conventional stent manufacturing method may be a manual operation method, takes a long time, lowers the productivity of the product, and increases manufacturing cost.

### DISCLOSURE

### Technical Problem

The present invention has been proposed in order to solve the above problems. The present invention provides a method of manufacturing a stent that is capable of producing the stent, inexpensively, quickly, simply, and without limitation of manufacturing sites. Further, a stent manufactured by the above method is proposed in accordance with the present invention.

### Technical Solution

One aspect of the present disclosure provides a stent comprising: a hollow tubular body portion; a hooking portion connected to one end of the body portion; and a hooked portion connected to the other end of the body portion, wherein the hooking portion is hooked on the hooked portion.

Another aspect of the present disclosure provides a method for manufacturing a hollow tubular stent, the method comprising: forming a flat stent structure in a two-dimensional shape having a thin thickness using 3D printing; and curling the flat stent structure into a hollow tubular form by at least one deformation factor.

### Advantageous Effect

According to the present disclosure, using the automated process, the stent may be manufactured inexpensively, quickly, simply, and without manufacturing location constraints.

### DESCRIPTION OF DRAWINGS

FIG. 1 is a perspective view of the stent before deformation according to an embodiment, and is a drawing immediately after 3D printing of the stent.

FIG. 2 is a perspective view of a first deformed stent with the hooking portion deformed.

FIG. 3 is a perspective view of a second deformed stent with the body portion deformed.

FIG. 4 is a perspective view of a third deformed stent that is inserted into the body and is subsequently expanded therein.

FIG. 5 is an enlarged view of a section A in FIG. 1.

FIG. 6 is an enlarged perspective view of the deformable portion.

FIG. 7 is a plan view of the deformable portion after a to-be-deformable portion is deformed.

FIG. 8 is an enlarged view of a section B in FIG. 2.

FIG. 9 is a cross-sectional view taken along a line I-I′ in FIG. 8.

FIG. 10 is an enlarged view of a section C in FIG. 2.

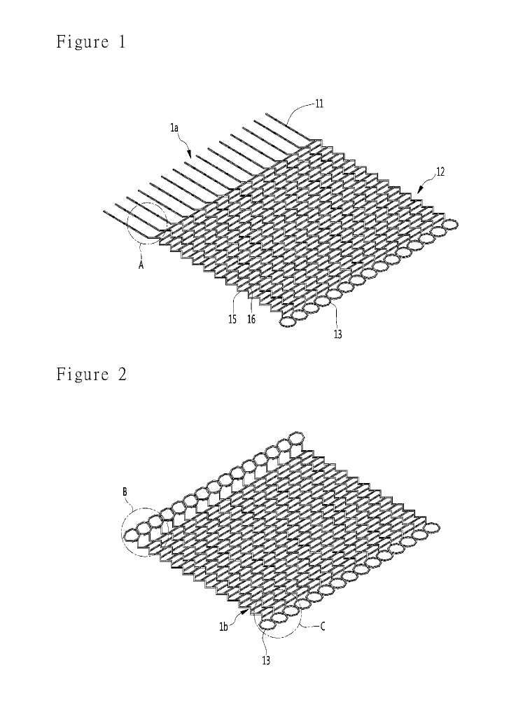

FIGS. 1i and 12 are views showing ends of the hooking portion 11 and the hooked portion 13.

FIG. 13 is a perspective view of the stent according to a third embodiment.

FIG. 14 is an enlarged view of the hooking portion of the stent according to a third embodiment.

FIG. 15 is a view showing the stent observed in the direction of the arrow (A direction) in FIG. 14.

FIG. 16 and FIG. 17 are enlarged views of the hooking portion and the hooked portion according to a fourth embodiment.

### MODE FOR INVENTION

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

The accompanying drawings are included to facilitate the understanding of the invention. In the drawings, in describing the overall structure, a minute portion may not be specifically expressed. The entire structure may not be specifically reflected in the description of the minute portion. In addition, even when specific details of the elements such as the installation positions thereof are different, the same names are given to the elements when the functions thereof are the same. Thus, the convenience of understanding may be enhanced. When there are a plurality of identical elements, only one element will be described, and the same description will be applied to the other elements, and a description thereof will be omitted.

Before describing the embodiment, 4D printing will be described.

In 4D printing, a smart material such as a shape memory alloy or a resin is printed using a 3D printer in a thin 2D shape to form a printed object. The printed object deforms to different desired shapes as time or the surrounding environment changes. A target shape and the target condition associated with the printed object deformation may be pre-programmed. In this connection, deformation factors may be diverse environments or sources of energy such as heat, vibration, gravity, moisture, light, and pH, etc. The 4D printing can shorten the long manufacturing time which was a big problem in conventional 3D printing. In this respect, the 4D printing will be an area where industrial use is highly expected.

### First Embodiment

The present disclosure features the manufacture of the stent using the 4D printing technique.

FIG. 1 is a perspective view of the stent before deformation according to an embodiment, and is a drawing immediately after 3D printing of the stent.

Referring to FIG. 1, the stent 1a before deformation is formed into a substantially two-dimensional plane. The stent 1a before deformation is printed in a substantial two-dimensional plane for its fast 3D printing. In practice, the pre-deformed stent has a predetermined height, but the pre-deformed stent is small in height for its rapid 3D printing and thus is printed in a substantially two-dimensional plane. Therefore, the pre-deformation stent may be regarded as equivalent to a two-dimensional plane. The hooking portion 11 and the hooked portion 13 are coupled to both ends of the stent 1a before deformation. A body portion 12 is provided as a portion connecting the hooking portion 11 and the hooked portion 13. When the overall height of the stent 1a may be observed in an elevation side view, the height is uniform and the stent may be substantially the 2D object.

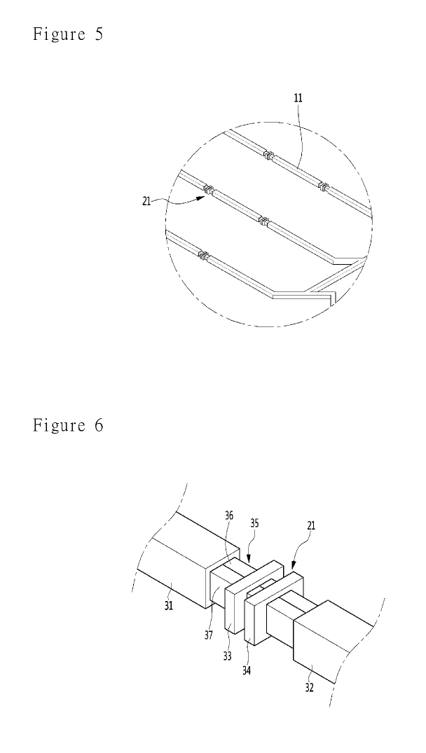

In detail, the hooking portion 11 may be embodied as a plurality of bars extending outwardly from the body portion 12. A number of bars may be connected to each other via a deformable portion (see 21 in FIG. 5). After the hooking portion 11 is deformed, at least a portion of the hooking portion 11 is hooked on the hooked portion 13, whereby the hooked portion 13 supports the hooking portion 11. For example, the hooked portion 13 may be provided as a plurality of rings. The number of bars and the number of rings may be the same. As a result, each bar may be hooked on each ring in correspondence with each other to maintain the deformed shape of the stent.

The body portion 12 may include a plurality of first directional extensions 15 connecting the hooking and hooked portions 11 and 13 and extending zigzag in the first direction; and second directional extensions 16 extending in a direction intersecting the extending direction of the first directional extensions 15 and connecting the first directional extensions 15 to each other. The first directional extension 15 extends in a zigzag manner. Thus, when the zigzag extension angle is large, the diameter of the stent can be increased. The first directional extension 15 is not limited to zigzag extension. The first directional extension 15 may extend into another shape, such as a deformable curve. However, as will be described later, in order to securely arrange the deformable portion 21, it is preferable that the first extending portion extends in a zigzag shape. The second directional extension 16 can maintain the overall shape of the stent. The number of the second directional extensions 16 may be provided as a number required for maintaining the shape of the stent. In one example, the number of extensions 16 may be one, two, or multiple. Also, since the rings forming the hooked portion 13 are connected to each other, the second directional extension 16 may not be provided. However, in order to maintain a stable stent shape, it is preferable that a plurality of second extensions 16 are provided.

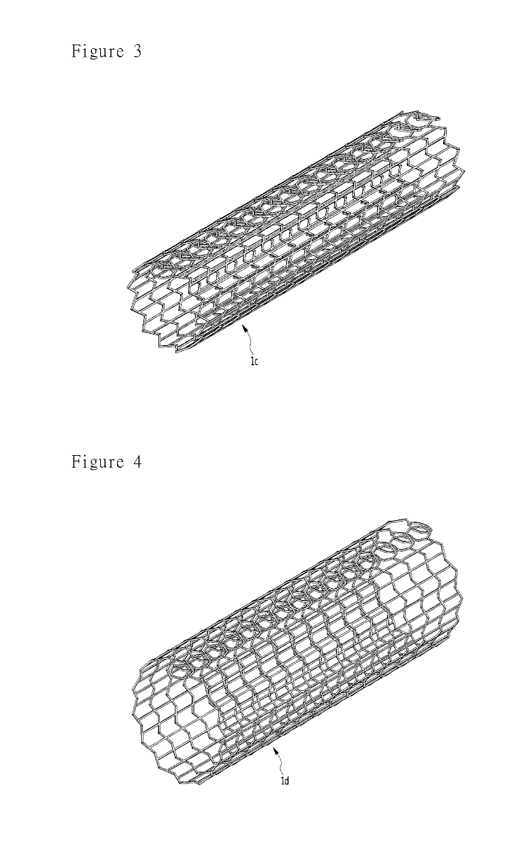

FIG. 2 is a perspective view of a first deformed stent with the hooking portion deformed. FIG. 3 is a perspective view of a second deformed stent with the body portion deformed. FIG. 4 is a perspective view of a third deformed stent that is inserted into the body and is subsequently expanded therein.

Referring to FIG. 2, if the first deformation factor by which the hooking portion 11 is deformable is applied thereto, the bar constituting the hooking portion 11 is deformed in a ring shape so that the 3D printed stent is deformed into the first deformed stent 1b. Referring to FIG. 3, if the second deformation factor by which the body portion 12 can be deformed is applied thereto, the body portion 12 is deformed in a round shape. Thus, the first deformed stent 1b may be deformed into the second deformed stent 1c. In this connection, the hook of the hooking portion 11 is hooked to the ring of the hooked portion 13, whereby the hooking and hooked portions 11 13 can be fastened to each other. Referring to FIG. 4, if the third deformation factor by which the body portion 12 is deformed is applied thereto, the body portion 12 is deformed to expand by itself. Thus, the second deformed stent 1c may be deformed to the third deformed stent 1d. The third deformed stent 1d may be regarded as a result of the stent being deformed after the stent is inserted into the body. The third deformed stent 1d is formed in an elongate tubular shape in one direction. In order to maintain the shape of the tube, the hooking and hooked portions 11 13 can be fastened to each other. In other words, the body portion is curled to form a tubular shape, and, then, the hooking and hooked portions 11 13 can be fastened to each other such that the tubular shape can be maintained.

The deformation order at which the deformation factors are applied thereto may be changed. For example, the second deformation factor by which the body portion 12 is curled is applied thereto first. Next, the first deformation factor, by which the hooking portion 11 is deformed in a ring shape, may then be applied thereto. In this connection, the end portion of the hooking portion 11 moves toward the hooked portion 13 by the curling operation of the body portion 12. Next, by the curling operation of the hooking portion 11, the hooking and hooked portions 11 13 may be fastened to each other such that the tubular shape can be maintained. The third deformation may be a final deformation performed after the stent is mounted on the damaged portion of the body.

Depending on stages of deformations, various deformation factors may be applied thereto sequentially or together. Hereinafter, deformation will be exemplarily described.

First, the first deformation factor and its related structure and contents will be described.

FIG. 5 is an enlarged view of a section A in FIG. 1.

Referring FIG. 5, the hooking portion 11 is embodied as a plurality of bars. The bar may be embodied in a shape extending in either direction. The bar may be provided with a plurality of deformable portions 21 at predetermined intervals. Alternatively, the deformable portion 21 may not be provided. The number of the deformable portions 21 may be configured such that the bar is deformed in an annular shape so that at least the end of the bar may be hooked on the ring constituting the hooked portion 13. In the present embodiment, seven of the portions 21 are equally spaced, but this is only an example.

The deformable portion 21 may be implemented using a smart material. The smart material refers to a material that may be deformed in a different form from the original shape when a predetermined deformation factor is applied thereto, as described above. In this connection, the deformation factor may include various environments or energy sources such as heat, vibration, gravity, moisture, light, and pH.

FIG. 6 is an enlarged perspective view of the deformable portion.

Referring to FIG. 6, the deformable portion 21 is disposed between the first bar 31 and the second bar 32. The first bar 31 and the second bar 32 constitute bars constituting the hooking portion 11. The deformable portion 21 comprises a smart material 35. The smart material 35 may include a first material 36 and a second material 37 arranged in a direction in which deformation occurs. The smart material 35 may be implemented as a single strand connecting the first bar 31 and the second bar 32 directly. Stoppers 33 and 34 may be fastened to the deformable portion 21. The stoppers 33 and 34 may be configured to suppress excessive deformation of the smart material 35 and to control the deformation angle. The stoppers 33 and 34 contact each other while the smart material 35 is deformed to control the deformation angle thereof. The stoppers 33 and 34 may contact the bars 31 and 32 respectively to limit the deformation angle of the deformable portion. In the figure, an embodiment is shown in which stoppers 33 and 34 contact each other to limit the deformation angle.

The smart material may be resin or metal. Illustratively, the smart material may be found in the paper “Stimuli responsive self-folding using thin polymer films” by David H Gracias, as published at www.sciencedirect.com. The smart material may be disclosed in Current Opinion in Chemical Engineering 2013, 2: 112-119. In this paper, a resin material is used as a smart material. When the deformation factor is applied thereto, a curvature deformation of the material is generated. Another smart material is disclosed at the paper “Curving Nanostructures Using Extrinsic Stress”, written by Jeong-Hyun Cho, Teena James, and David H. Gracias, as published at www.advmat.de. This smart material is described in Adv. Mater. 2010, 22, 2320-2324. In this paper, Sn and Ni metal materials are used as smart materials. When the deformation factor is applied thereto, a bent deformation occurs. It is to be understood that the smart material is not limited to the materials presented in the papers.

As described above, the smart material 35 is not limited to metal or resin. By using the two materials 36 and 37, the smart material may be deformed when a particular deformation factor is applied thereto. Again, the smart material and the deformation factor are not limited to those described herein. Any smart material and deformation factor that may lead to deformation thereof may be employed. However, the deformable portion 21 is processed by 3D printing into a shape that is strictly three-dimensional but substantially two-dimensional. If the deformation factor is applied thereto, the deformable portion 21 may be deformed. This is called 4D printing.

The deformable portion is very small and may not be displayed in other figures. However, it may be easily guessed that the deformable portion appears in the right place in this detailed descriptions.

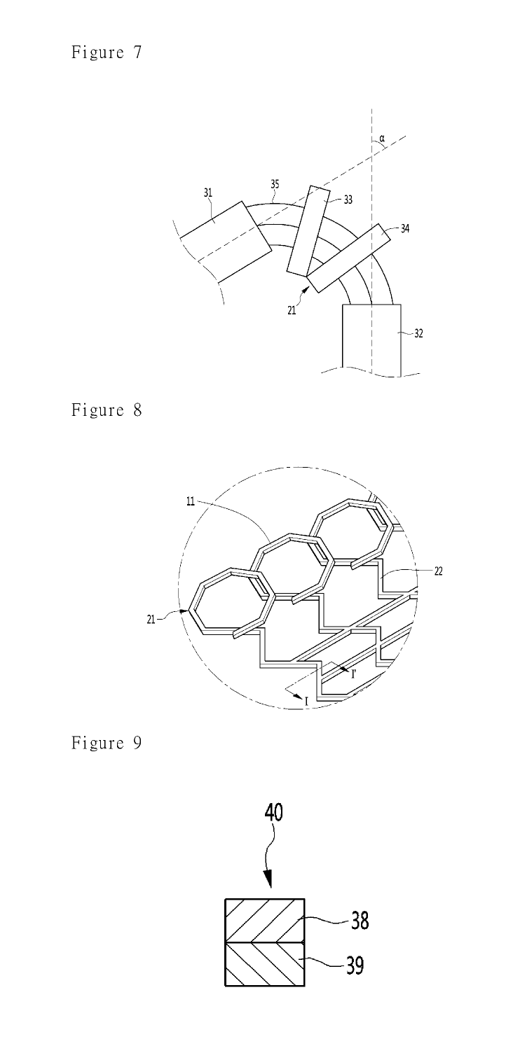

FIG. 7 is a top view of the deformable portion after the deformable portion is deformed.

Referring to FIG. 7, if the specific deformation factor that can cause the deformation of the deformable portion 21 is applied thereto, the smart material 35 is deformed. In one example of a form in which the smart material 35 is deformed, the first material 36 may be expanded relatively more than the second material 37. In this case, the deformation of the smart material 35 is executed such that a curvature center is formed on the second material 37. The smart material 35 is bent and deformed. Although only one smart material 35 implemented by the deformable portion 21 is described, the same description may be applied to other smart materials 35 as well. In particular, the smart material 35 disposed on both sides of the stoppers 33 and 34, and the smart material 35 disposed between the stoppers 33 and 34 may also be bent and deformed.

In order to prevent the deformable portion 21 from being excessively bent, stoppers 33 and 34 are implemented. Hereinafter, the operation of the stoppers 33 and 34 will be described in more detail. The stoppers 33 and 34 allow deformation of the deformable portion 21 to be adjusted to a predetermined degree when the smart material 35 is deformed. That is, the stoppers can prevent deformation from being deformed beyond a predetermined degree. As shown in the figure as an exemplary operation, when the stoppers 33 and 34 contact each other, even though the smart material 35 disposed between the pair of stoppers 33 and 34 tries to more deform, the material 35 is no longer deformed since the material 35 contacts the stoppers 33 and 34. Therefore, due to the contact between the stoppers 33 and 34, the deformation limit angle α of the smart material 35 may be realized.

In the drawings, it is described that the deformation limitation may be controlled only by the contact between the stoppers 33 and 34, but the present invention is not limited thereto. For example, the deformation limit may be controlled by either one of stoppers 33 and 34 touching first bar 31 or second bar 32.

In order to prevent breakage of the smart material 35, when the deformation factor is applied thereto, it may be desirable that the amount of deformation of the smart material 35 itself becomes a degree slightly exceeding the deformation limitations imposed by the stoppers 33 and 34. This is because if the amount of deformation of the smart material 35 itself significantly exceeds the deformation limitations imposed by the stoppers 33 and 34, strong stress is generated in the vicinity of the smart material 35, and the smart material 35, and, thus, the smart material may be damaged. To the contrary, if the amount of deformation of the smart material 35 itself is smaller than the deformation limitation limited by the stoppers 33 and 34, a sufficient amount of deformation cannot be achieved.

FIG. 8 is an enlarged view of a section B in FIG. 2.

Referring to FIG. 8, when each deformable portion 21 is deformed, it may be seen that the bars constituting the hooking portion 11 are deformed to form a polygon with its respective deformable portions 21 acting as vertexes of the polygon. The end of the bar forming the hooking portion 11 may be deformed into a ring shape. As will be explained in more detail later, the end of the bar is hooked to the ring of the hooked portion, thereby, the fastening structure between the hooking and hooked portions 11 and 13 may be maintained.

By this action, the first deformed stent may be completed.

FIG. 9 is a cross-sectional view taken along a line I-I′ in FIG. 8.

FIG. 9 is a cross-sectional view of the first directional extension 15 that implements the body portion 12. The first directional extension 15 may use a smart material 40. The upper portion of the smart material 40 may use the first material 39 and the lower portion thereof may use the second material 38. The smart material may be deformed when deformation factor is applied thereto. The deformation factor may include various environments or energy sources such as heat, vibration, gravity, moisture, light, and pH. Therefore, when a deformation factor corresponding to the smart material 40 is applied thereto, deformation occurs. The deformation may be implemented in the form of a bend so that the radius of curvature lies on the second material 38. In other words, the smart material 40 may be deformed in such a way that the first material 39 is relatively more elongated than the second material 38. The deformation factor for the body portion 12 may be the same or different from the deformation factor for the hooking portion 11. For that reason, the portions 12 and 11 have different reference numerals.

The above-described deformation method is a bending method of forming the deformable portion by forming a deformable portion as double materials. However, this embodiment is not limited to such a bending method. For example, when one polymer material is used instead of dual materials, bending of the hooking portion and the hooked portion may be performed by varying the crossing-linking degree based on the thickness or the side dimension.

Such a deformation may be implemented as follows. The body portion 12 is curled so that the hooking portion 11 approaches the hooked portion 13 and the bar of the hooking portion is hooked into the ring of the hooked portion. By this action, a second deformed stent may be completed.

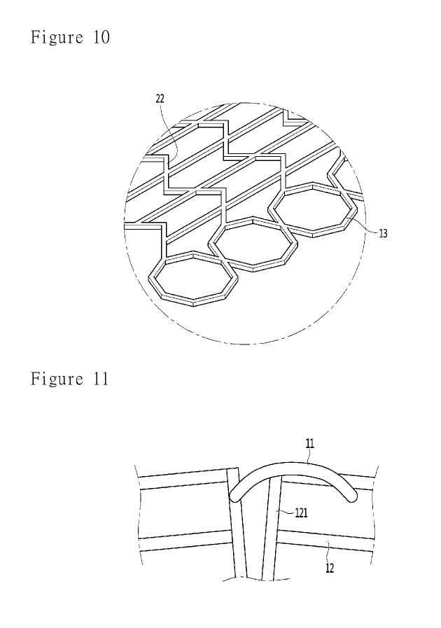

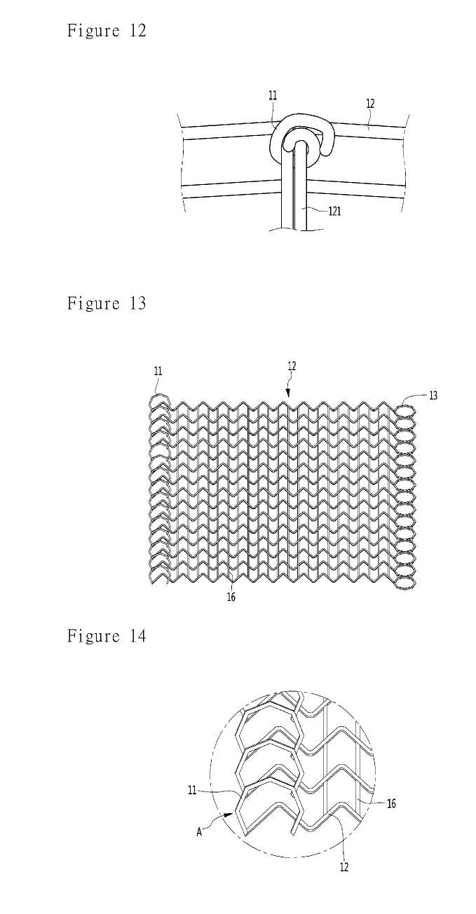

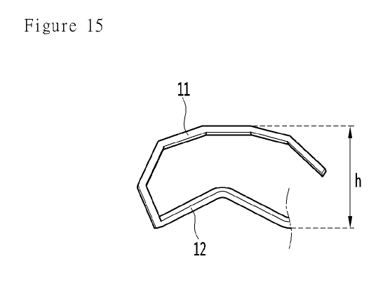

FIG. 10 is an enlarged view of a section C of FIG. 2. FIG. 10 shows a ring constituting the hooked portion 13. However, it is not limited to the shape of the ring. The hooked portion may be embodied as a hook or a ring or a hook in a manner similar to the hooking portion 11.

Once the second deformed stent 1c is completed, the manufacture of the stent for sale may be considered to be completed. This is because the stent that is inserted into the body is provided to have a narrow diameter, and must be expanded after being inserted into the body.

Deformation of the second deformed stent 1c to the third deformed stent 1d means that the second deformed stent 1c is inserted into the body and expanded using an artificial expanding tool such as a balloon. Also, in one embodiment, at the time of deformation of the stent in the body, an artificial expansion tool for the stent may not be used. The use of an artificial expanding device is not required when there is no need for additional expansion in the body and when self-expansion may occur due to body temperature and moisture in the body.

In order to stably expand the stent during expansion and to secure the retaining force of the stent, the first directional extension 15 may be implemented in a zigzag form. In addition, a deformable portion may be disposed at the zigzag bent portion of the first directional extension 15. Thereby, the angle of the bent portion may be increased by controlling the smart material so that the angle of the deformable portion is increased. In this case, on the whole, the diameter of the second deformed stent 1c is expanded to form the third deformed stent 1d. This allows the stent to secure the diameter of the hollow channel in the body.

### Second Embodiment

The configurations and operations of the hooking and hooked portions of the second embodiment and the hooking and hooked portions 11 and 13 of the first embodiment are different from each other. Therefore, the description of the first embodiment is applied to portions without a specific description in the second embodiment. The detailed description of the first embodiment shall be applied to the description of the overlapping portions of the second embodiment.

FIG. 11 and FIG. 12 show the ends of the hooking portion 11 and the hooked portion 13. The body portion 12 is shown in the drawings. FIG. 11 shows a state before the first deformation factor by which the hooking portion 11 is deformed is applied thereto. FIG. 12 is a diagram showing a state after the first deformation factor is applied thereto.

Referring to FIG. 11 and FIG. 12, when the second deformation factor is met. The body portion 12 is first curled. Thereafter, if the first deformation factor is applied thereto, the hooking portion 11 is deformed. The portion 11 is securely hooked to the hooked end 121 of the hooked portion 13. In this connection, the hooking portion 11 has a plurality of deformable portions, so that the portion 11 may be hooked on the hooked end 121 more reliably. In the figure, the hooking portion 11 is wound around the hooked portion 12, more precisely the hooked end 121, by 3/2 turns. However, the present disclosure is not limited thereto. The hooking portion 11 is wound around the hooked portion 12, more precisely the hooked end 121, by a number larger than 3/2 turns. In addition, the hooking portion 11 may be hooked on the end of the body portion 12 without the hooked portion 13.

### Third Embodiment

The configuration and operation of the hooking portion of the second embodiment, and the configurations and operations of the hooking portions of the first and second embodiments are different from each other. Therefore, the description of the first and second embodiments is applied to portions without a specific description in the third embodiment. The detailed description of the first embodiment and second embodiment shall be applied to the description of the overlapping portions of the second embodiment.

FIG. 13 is a perspective view of the stent according to a third embodiment. FIG. 14 is an enlarged view of the hooking portion of the stent according to a third embodiment. FIG. 15 is a view showing the stent observed in the direction of the arrow (A direction) in FIG. 14.

Referring to FIG. 13 to FIG. 15, when printing the hooking portion 11, its material may be varied according to the height of the hooking portion 11, similar to the first directional extension 15. Specifically, when observing in the thickness direction, the hooking portion 11 may be printed so that the lower material is relatively more extended than the upper material. That is, the hooking portion 11 may be printed using two smart materials. Then, when the hooking portion 11 is deformed, the end of the hooking portion 11 may be lifted by h in comparison with the first directional extension 15. In this case, the fastening between the hooking and hooked portions 11 and 13 is performed reliably. The deformation factor is called a fourth deformation factor.

Other examples that may be included in the scope of the present disclosure will be set forth. First, the second directional extension 16 may not be provided or its number may be limited. For example, when sufficient strength may be secured by connecting the bars or rings of hooking or hooked portions 11 and 13 together, the second directional extension 16 may not be provided. However, in order to secure the strength of the stent, it may be more desirable to provide a second directional extension 16. Also, the deformation of the hooking and hooked portions 11 and 13 may be deformed in various directions, not only in the vertical and/or horizontal directions. For example, when the properties of a single material change in multiple directions, various materials may be combined in various orientations, 3D printing may done in many directions, the deformation of the hooking and hooked portions 11 and 13 may be deformed in various directions. However, the present invention is not limited thereto.

FIG. 16 and FIG. 17 are enlarged views of the hooking portion and the hooked portion according to a fourth embodiment. Referring to FIG. 16, the hooking portion 11 of the bar shape is deformed to enter the inside of the hooked portion 13 in the form of a ring. Thereafter, FIG. 17, the hooking portion 11 and the hooked portion 13 are fastened together. This type of fastening may be divided into the following three cases.

First, the ring of the hooked portion 13 is contacted and engaged with the wire or hook of the hooking portion 11 while the ring being reduced in the direction of the inner central axis. Secondly, a wire or hook of the hooking portion 11 is expanded outwardly in contact with and engaged with the ring or ring of the hooked portion 13. Thirdly, the ring of the hooked portion 13 is contacted and engaged with the wire or hook of the hooking portion 11 while the ring being reduced in the direction of the inner central axis, at the same time, a wire or hook of the hooking portion 11 is expanded outwardly in contact with and engaged with the ring or ring of the hooked portion 13.

The deformation of the hooking portion 11 and the hooked portion 13 may be presented in various cases based on the difference in shape and structure of the stent.

### INDUSTRIAL AVAILABILITY

According to the present disclosure, the stent may be manufactured by 4D printing method. Accordingly, the stent may be manufactured in an automated process at low cost, expeditiousness, simplicity, and no manufacturing site constraints.

1. A stent comprising:
a hollow tubular body portion;
a hooking portion connected to one end of the body portion; and
a hooked portion connected to the other end of the body portion, wherein the hooking portion is hooked on the hooked portion.

2. The stent of claim 1, wherein the hooking portion comprises a connection of at least two bars, wherein the hooking portion includes a deformable portion formed at a boundary between the at least two bars, wherein the deformable portion is made of a smart material which is deformed when a first deformation factor is applied thereto.

3. The stent of claim 1, wherein the hooking portion includes a ring, wherein the hooked portion includes a ring or hook or a bar.

4. The stent of claim 1, wherein the body portion includes at least two first directional extensions extending in a direction connecting the hooked portion and the hooked portion.

5. The stent of claim 4, wherein the body portion further comprises at least one second directional extension extending in a direction intersecting the extending direction of the at least two first directional extensions.

6. The stent of claim 4 wherein each first directional extension is stretchable.

7. The stent of claim 4, wherein each first directional extension includes a deformable portion of a smart material that is deformed when a third deformation factor is applied thereto.

8. The stent of claim 2 wherein the deformable portion includes a stopper configured to limit a degree of deformation of the deformable portion by the smart material.

9. The stent of claim 4, wherein each first directional extension is made of a smart material curled when a second deformation factor is applied thereto.

10. The stent of claim 1, wherein the body portion, the hooking portion, and the hooked portion are integrally manufactured by a single manufacturing process.

11. The stent of claim 10, wherein the manufacturing process is a 3D printing process.

12. A method for manufacturing a hollow tubular stent, the method comprising:
forming a flat stent structure in a two-dimensional shape having a thin thickness using 3D printing; and
curling the flat stent structure into a hollow tubular form by at least one deformation factor.

13. The method of claim 12, wherein the at least one deformation factor includes:
a first deformation factor to enable an end of the flat stent structure to be deformed to form a hooking portion; and
a second deformation factor to enable the flat stent structure to be curled into a hollow tubular form.

14. The method of claim 13, wherein the at least one deformation factor includes a third deformation factor to enable a diameter of the tubular stent to increase.

15. The method of claim 13, wherein the at least one deformation factor includes a fourth deformation factor to enable the hooking portion to be lifted up.

16. The stent of claim 5 wherein each first directional extension is stretchable.

17. The stent of claim 7 wherein each deformable portion includes a stopper configured to limit a degree of deformation of the deformable portion by the smart material.
